(19) United States
(12) Patent Application Publication
(10) Pub. No.: US 2013/0011908 A1
(43) Pub. Date: Jan. 10, 2013

(54) Culturing Medium

(76) Inventor: Abdulsamad Ishrath, Orlando, FL (US)

(21) Appl. No.: 13/542,199

(22) Filed: Jul. 5, 2012

Related U.S. Application Data
(60) Provisional application No. 61/504,525, filed on Jul. 5, 2011.

(51) Int. Cl. C12N 1/20; C12M 1/22

(52) U.S. Cl. 435/253.6; 435/305.1

(57) ABSTRACT

Herein disclosed is a culturing media, having at least one agar material and a swarming inhibiting quantity of anti-swarming compound wherein the ratio of the at least one agar material relative to the quantity of the anti-swarming compound is such to ameliorate cellular swarming by a first microbe and increasing cellular growth rate of a second microbe relative to a swarm rate of the first microbe. The culturing media may have at least one nutrient. The culturing media may be used to prevent swarming of certain strains of cells, or it may be used as a regular culturing media.

### CROSS REFERENCE TO RELATED APPLICATIONS

This application claims priority to U. S. Provisional Application No. 61/504,525 filed Jul. 5, 2011 which is incorporated herein in its entirety

### FIELD

The disclosure relates to culturing mediums in general, and in more specific examples, to anti-swarming culturing media.

### BACKGROUND

In microbiology, cell culturing is a common task. In many cases, the cell culturing may be aimed at growing and analyzing bacterial cell cultures. Sometimes, technicians may use a smearing technique whereby multiple suspected organisms will be applied to a culturing medium on a plate, such as an agar based medium, in a streaking pattern such that there may be multiple types of cells present in different locations on a single plate of culturing media. In certain cases, one strain may swarm at least a large portion of an agar plate and contaminate the other strains, providing the microbiologist with incorrect data. “Swarming” is a well-documented phenomenon and presents challenge in growing and analyzing bacterial via cell culture techniques (Dale Kaiser, “Bacterial Swarming: A Re-examination of Cell-Movement Patterns. Current Biology, Volume 17, Issue 14, R561-570, Elsevier Ltd, 17 Jul. 2007). What is therefore desired is a culturing medium that greatly inhibits swarming without inhibiting overall cellular growth.

### SUMMARY

Herein disclosed is a culturing media, having at least one agar material and a swarming inhibiting quantity of anti-swarming compound wherein the ratio of the at least one agar material relative to the quantity of the anti-swarming compound is such to ameliorate cellular swarming by a first microbe and increasing cellular growth rate of a second microbe relative to a swarm rate of the first microbe when the first and second microbes are grown on a culturing media made of the culturing media powder. The culturing media may have at least one nutrient. The culturing media may be used to prevent swarming of certain strains of cells, or it may be used as a regular culturing media.

Further disclosed is a slurry for making a culturing media. The term “slurry” as used herein is used in its broader sense of referring to a mixture of components in a solvent, typically the solvent being water. Still further disclosed is a culturing apparatus comprising at least one container and having at least a portion of an anti-swarming culturing media disposed on at least a portion thereof.

### BRIEF DESCRIPTION OF THE DRAWINGS

Some embodiments are better understood by reading the following Detailed Description, taken together with the Drawings, wherein,

FIG. 1 is an example of swarming tendencies of Proteus vulgaris after 24 hours on centrally inoculated control medium of Tryptic Soy Agar;

FIG. 2 is an example of swarming tendencies of Proteus vulgaris after 24 hours on a centrally inoculated embodiment in accordance with this Disclosure;

FIG. 3 is an example of swarming tendencies of Proteus vulgaris after 24 hours on another centrally inoculated embodiment in accordance with this Disclosure;

FIG. 4 is an example of swarming tendencies of Proteus vulgaris after 24 hours on another centrally inoculated embodiment in accordance with this Disclosure;

FIG. 5 is an example of swarming tendencies of Proteus vulgaris after 24 hours on another centrally inoculated embodiment in accordance with this Disclosure;

FIG. 6 is an example of swarming tendencies of Proteus mirabilis after 24 hours on centrally inoculated control medium of Tryptic Soy Agar; versus centrally inoculated embodiments in accordance with this Disclosure;

FIG. 7 is an example of swarming tendencies of Proteus mirabilis after 24 hours on a centrally inoculated embodiment in accordance with this Disclosure;

FIG. 8 is an example of swarming tendencies of Proteus mirabilis after 24 hours on another centrally inoculated embodiment in accordance with this Disclosure;

FIG. 9 is an example of swarming tendencies of Proteus mirabilis after 24 hours on another centrally inoculated embodiment in accordance with this Disclosure;

FIG. 10 is an example of swarming tendencies of Proteus mirabilis after 24 hours on another centrally inoculated embodiment in accordance with this Disclosure;

FIG. 11 is an example of swarming tendencies of Proteus vulgaris in a mixed culture including Bacillus cereus after 24 hours on a streak inoculated Tryptic Soy Agar control medium;

FIG. 12 is an example of swarming tendencies of Proteus vulgaris in a mixed culture including Bacillus cereus after 24 hours on a streak inoculated embodiment in accordance with this Disclosure;

FIG. 13 is an example of swarming tendencies of Proteus vulgaris in a mixed culture including Bacillus cereus after 24 hours on another streak inoculated embodiment in accordance with this Disclosure;

FIG. 14 is an example of swarming tendencies of Proteus vulgaris in a mixed culture including Staphylococcus aureus after 24 hours on a streak inoculated Tryptic Soy Agar control medium;

FIG. 15 is an example of swarming tendencies of Proteus vulgaris in a mixed culture including Staphylococcus aureus after 24 hours on a streak inoculated embodiment in accordance with this Disclosure;

FIG. 16 is an example of swarming tendencies of Proteus vulgaris in a mixed culture including Staphylococcus aureus after 24 hours on another streak inoculated embodiment in accordance with this Disclosure;

FIG. 17 is an example of swarming tendencies of Proteus mirabilis in a mixed culture including Staphylococcus aureus after 24 hours on a streak inoculated Tryptic Soy Agar control medium;

FIG. 18 is an example of swarming tendencies of Proteus mirabilis in a mixed culture including Staphylococcus aureus after 24 hours on a streak inoculated embodiment in accordance with this Disclosure;

FIG. 19 is an example of swarming tendencies of Proteus mirabilis in a mixed culture including Staphylococcus aureus after 24 hours on another streak inoculated embodiment in accordance with this Disclosure;

FIG. 20 is an example of swarming tendencies of Proteus vulgaris after 40 hours of central inoculation on different culture medium embodiments;

FIG. 21 is an example of swarming tendencies of Proteus vulgaris after 40 hours of 4-zone streaking inoculation on different culture medium embodiments;

FIG. 22 is an example of swarming tendencies of Proteus mirabilis after 40 hours of central inoculation on different culture medium embodiments;

FIG. 23 is an example of swarming tendencies of Proteus mirabilis after 40 hours of 4-zone streaking inoculation on different culture medium embodiments;

FIG. 24 is an example of swarming tendencies of Alcaligenes faecalis after 40 hours of central inoculation on different culture medium embodiments;

FIG. 25 is an example of swarming tendencies of Alcaligenes faecalis after 40 hours of 4-zone streaking inoculation on different culture medium embodiments;

### DETAILED DESCRIPTION

Disclosed embodiments in this Disclosure are described with reference to the attached figures, wherein like reference numerals are used throughout the figures to designate similar or equivalent elements. The figures are not drawn to scale and they are provided merely to illustrate the disclosed embodiments. Several aspects are described below with reference to example applications for illustration. It should be understood that numerous specific details, relationships, and methods are set forth to provide a full understanding of the disclosed embodiments. One having ordinary skill in the relevant art, however, will readily recognize that the subject matter disclosed herein can be practiced without one or more of the specific details or with other methods. In other instances, well-known structures or operations are not shown in detail to avoid obscuring structures or operations that are not well-known. This Disclosure is not limited by the illustrated ordering of acts or events, as some acts may occur in different orders and/or concurrently with other acts or events. Furthermore, not all illustrated acts or events are required to implement a methodology in accordance with this Disclosure.

Notwithstanding that the numerical ranges and parameters setting forth the broad scope of this Disclosure are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all sub-ranges subsumed therein.

In at least some embodiments, a culturing media powder is herein disclosed. The culturing media powder may be used to form a culturing medium. In some embodiments, the culturing medium may be used as an anti-swarming culturing medium.

The culturing media powder may have at least one agar material. The at least one agar material may be a solidifying agent effective to help solidify a liquid mixture into a solid or gelatinous mixture. In some embodiments, the at least one agar material may be a substantially pure agar such as Bacto Agar sold by DIFCO. The comparison of Bacto Agar and the additional suitable solidifying agents can be studied by the following table.

### Few Important Physical Properties & Applications.

| | Clarity/ | | |
|---|---|---|---|
| | Transparency | | |
| | (NTU)* | Gel | |
| | Nephelometric | Strength | |
| Type Of Agar | Turbidity Units | (g/cm²) | Applications. |
| 1. Bacto Agar | 4.3 | 600 | Microbiological |
| | | | Culture Media |
| 2. Difco | 5.3 | 560 | Same as above |
| AgarGranulated | | | ( |
| 3. BBL Agar | <10 | 600-800 | Same as above |
| Grade ‘A’ | | | (Contains essential |
| | | | minerals) |
| 4. Difco Agar | 26.2 | 613 | Same as above |
| Technical | | | (not highly processed) |
| 5. BBL Agar | N/A | N/A | Used in Molecular |
| Select | | | Genetics |
| 6. BBL Agarose | <10 | 800-1200 | Used in Gel |
| | | | Electrophoresis |
| 7. Difco Agar | 3.7 | 700 | Used in mammalian & |
| Noble | | | plant tissue culture, |
| | | | immunodiffusion, |
| | | | & Electrophoresis. |

Note:
Based on the usage and ‘clarity value’ of the agars (1-4), plates poured with Bacto Agar are more transparent which makes them easier to look through and decreases the risk of contamination or condensed water which smears the plate.

In at least some embodiments, the culturing media powder may have at least one nutrient. The at least one nutrient may be any cellular nutrient that facilitates cellular growth on a culturing medium. In some embodiments, the at least one nutrient is enzymatic (e.g. papaic) digest of soybean, digest of casein (e.g. pancreatic digest), or any combination thereof. Examples of other nutrients include, but are not limited to, enzymatic digests of animal proteins from muscular tissues, waste parts, bones and cartilage, infusions from calf brain and beef heart. Yeast extract may also be used as a nutrient source.

In at least some embodiments, the culturing media powder may have at least one swarming inhibiting quantity of an anti-swarming compound. The anti-swarming compound may be a salt, for example. In at least some embodiments, the anti-swarming compound may be sodium chloride (NaCl).

In at least some embodiments, the culturing media powder may have at least one agar material, at least one nutrient and at least one swarming inhibiting quantity of an anti-swarming compound. The ratio of the at least one agar material relative to the quantity of the anti-swarming compound may be such to ameliorate cellular swarming by a first microbe. The ratio may also be such to have an increase in cellular growth rate of a second microbe relative to a swarm rate of the first microbe when the first and second microbes are grown on a culturing media made of the culturing media powder.

In at least some embodiments, the ratio of ‘the at least one anti-swarming compound to the at least one agar material to the at least one nutrient is about 2.5-3.5: about 2.5-3.5: about 2.5-3.5, respectively. The term about in reference to percentage values or ratios is to mean approximately 5% plus or minus the value.

In some embodiments, the at least one anti-swarming compound may be present in an amount ranging from about 20% by weight to about 40% by weight of the culturing media powder and in a ratio of about 1-3: about 1-5 of the at least one anti-swarming compound to the at least one agar material, respectively.

For convenience, as used throughout, solution refers to, but is not limited to, a water or water-based liquid. The culturing media powder may be such that a weight of powder per volume solution dose (w/v dose) of the culturing media powder is effective to provide a swarming inhibiting quantity of the at least one anti-swarming compound ranging from about 1-4% (w/v). In some embodiments, the w/v dose of the culturing media powder is effective to provide a swarming inhibiting quantity of the at least one anti-swarming compound in an amount of about 1.5% w/v.

The w/v dose may also be effective to provide a quantity of the at least one agar material in an amount of about 1.5-3% w/v. The w/v dose may also be effective to provide at least one nutrient in an amount of about 2% w/v. In a more specific embodiment, a papaic digest of soybean is provided in an amount of about 0.5% w/v of a water solution; the w/v dose may also be effective to provide a pancreatic digest of casein in an amount of about 1.5% w/v.

In some embodiments, a w/v dose may be effective to provide a quantity of anti-swarming compound in an amount of about 1.5% w/v, an agar material in an amount of about 2.5% w/v, a papaic digest of soybean in an amount of about 0.5% w/v and a pancreatic digest of casein in an amount of about 1.5% w/v

In some embodiments, the w/v dose is about 60 grams of the culturing media powder per liter of the water solution.

Further described herein is culturing media slurry for making a culturing medium and having at least some culturing media powder as described above mixed with a liquid such as water. The slurry may be an anti-swarming culturing media slurry and may be used to make an anti-swarming culturing medium.

The anti-swarming culturing media slurry may have at least one agar material as described above, at least one nutrient as described above, a swarming inhibiting quantity of at least one anti-swarming compound as described above, and a quantity of water.

A ratio of the at least one agar material relative to the quantity of the anti-swarming compound may be such to ameliorate cellular swarming by a first microbe and increasing cellular growth rate of a second microbe relative to a swarm rate of the first microbe when the first and second microbes are grown on a culturing media made at least partly of the culturing media slurry.

The first microbe may be any microbe that tends to swarm over a culture medium. In some embodiments, the first microbe may be Proteus vulgaris, Proteus mirabilis, Alcaligens faecalis, or any combination thereof.

The second microbe may be any other microbe the generally becomes swarmed by a first microbe as described above when on a shared culturing medium. For example, the second microbe may be selected from but not limited to Staph.aureus, Staph.epidermdis, Staph.sapro, Strep.agalacteae, Strep.pyogenes, Strep.bovis, E. coli, Salmonella sp., Shigella sonii, Enterobactor aerogenes, Bacillus cereus, Pseudomonas sps., Serratia m., Salmonella typhimurium, Cornybacteriuma diphtheria(avirulent), Providencia rettgeri, and Citrobacter frundii, and any combination thereof.

In some embodiments, the anti-swarming compound is present in the slurry in an amount ranging from about 10 g/L to about 40 g/L of solution. In some embodiments, the anti-swarming compound is present in an amount of about 15 g/L of solution.

The at least one agar material may be present in the slurry in an amount ranging from about 15 g/L to about 30 g/L of solution. In some embodiments, the at least one agar material may be present in an amount of about 25 g/L of solution.

The at least one nutrient may comprise papaic digest of soybean in an amount of about 5 g/L of solution and pancreatic digest of casein in an amount of about 15 g/L of solution.

In some embodiments, the culturing media slurry may have the at least one anti-swarming compound present in an amount of about 15 g/L of solution, the at least one agar material present in an amount of about 25 g/L of solution, papaic digest of soybean in an amount of about 5 g/L of solution, and pancreatic digest of casein in an amount of about 15 g/L of solution.

Further described is a culturing medium made from any of the above described culturing media powder or culturing media slurry embodiments.

Still further described is a culturing apparatus for preventing swarming, having at least one container and an anti-swarming culturing medium disposed on at least a portion of at least one dish. The anti-swarming culturing media may be made from an anti-swarming culturing media powder and/or slurry as described above. The at least one container may be anything effective to contain at least a portion of said anti-swarming culturing medium such as but not limited to at least one petri dish, plate or the like, at least one test tube or the like, or any combination thereof.

Even further described is a method for making an anti-swarming culturing media comprising providing a culturing media powder as described above, mixing the powder with water to create a slurry, and allowing said slurry to form a gelatinous solid on at least one surface.

### EXAMPLES

### Example 1

Several embodiments of the above disclosed culturing medium were tested for cellular growth and swarming. Proteus vulgaris ATCC# 43071 and Proteus mirabilis ATCC# 8427 were grown separately in tryptic soy broth at 35 degrees Celsius for 20 hours.

Four non-limiting examples of embodiments of culturing media were created by modifying DIFCO Tryptic Soy Agar (TSA) with NaCl and DIFCO Bacto Agar (DBA) in solution of water. The four non-limiting examples are culturing media B (cmB), culturing media A (cmA), culturing media A-1 (cmA-1), culturing media A/1 (cmA/1). The composition of the media are as follows:

cmB=TSA+0.256 mM/ml NaCl+0.01 g/ml DBA

cmA=TSA+0.256 mM/ml NaCl+0.005 g/ml DBA

cmA-1=TSA+0.17 mM/ml NaCl+0.005 g/ml DBA

cmA/1=TSA+0.17 mM/ml NaCl+0.01 g/ml DBA

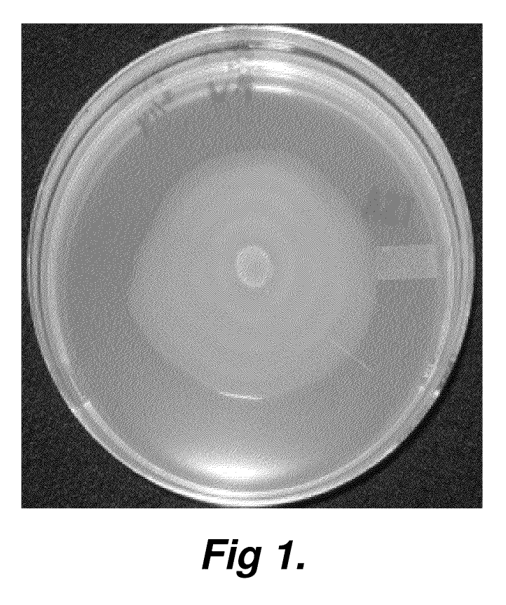

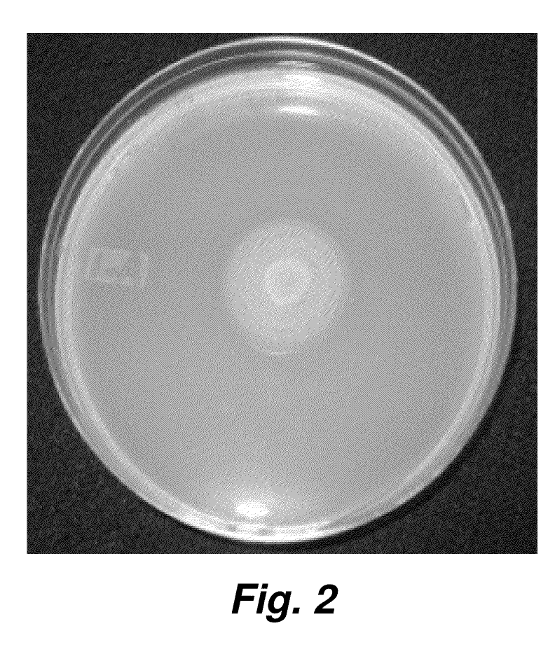

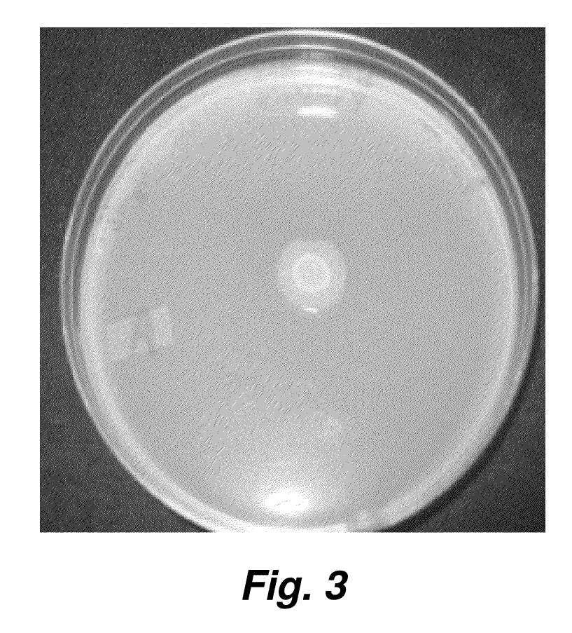

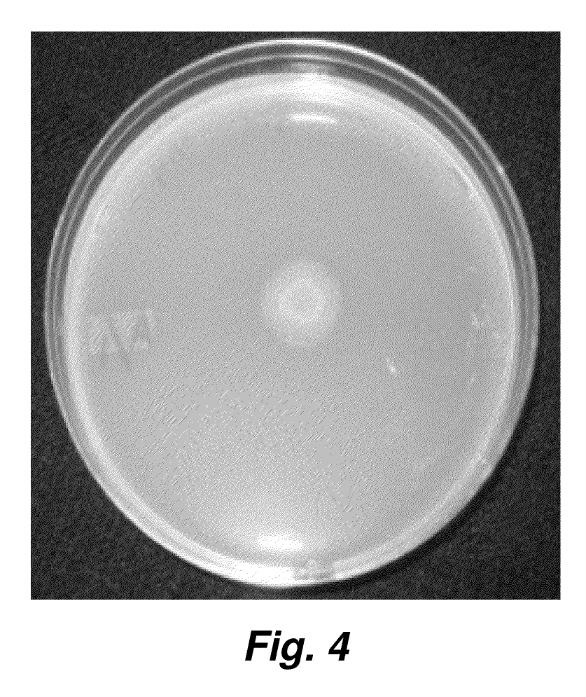

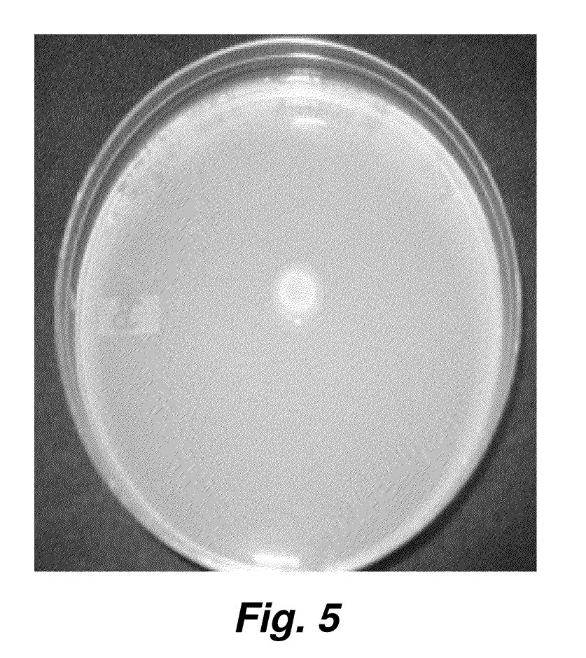

Referring to FIG. 1 through FIG. 5, 5 μL of Proteus vulgaris was inoculated centrally on five separate plates containing a pure TSA control plate and the four tested culturing media and given 24 hours to grow. FIG. 1 shows an example of swarming tendency on TSA. FIG. 2 shows swarming tendency on cmA-1. FIG. 3 shows swarming tendency on cmA. FIG. 4 shows swarming tendency on cmA/1. FIG. 5 shows swarming tendency on cmB. Swarming is clearly reduced on all tested embodiments.

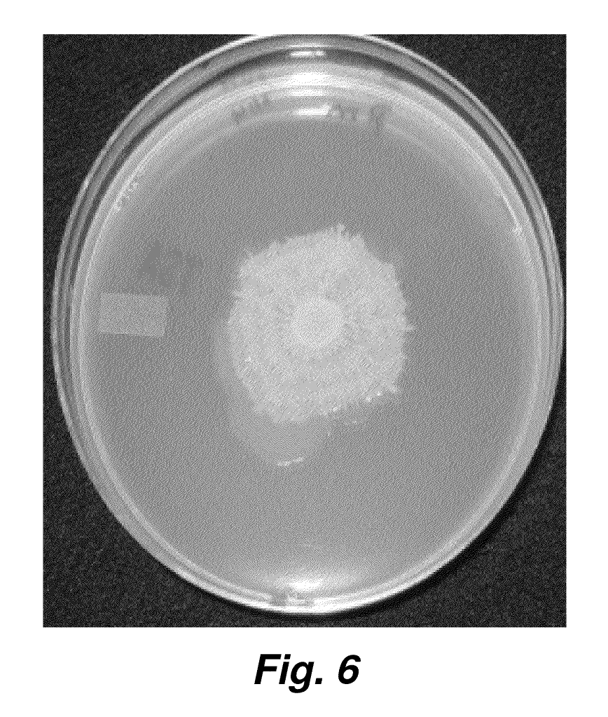

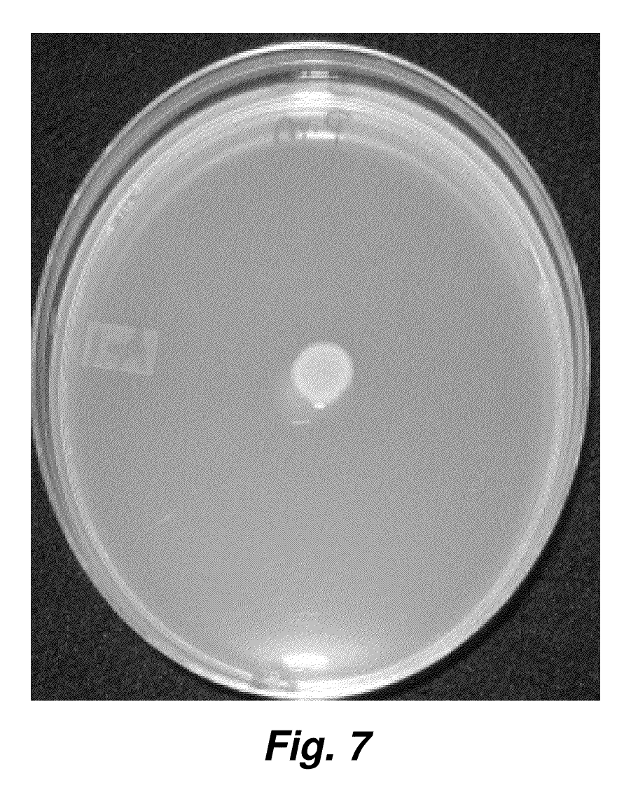

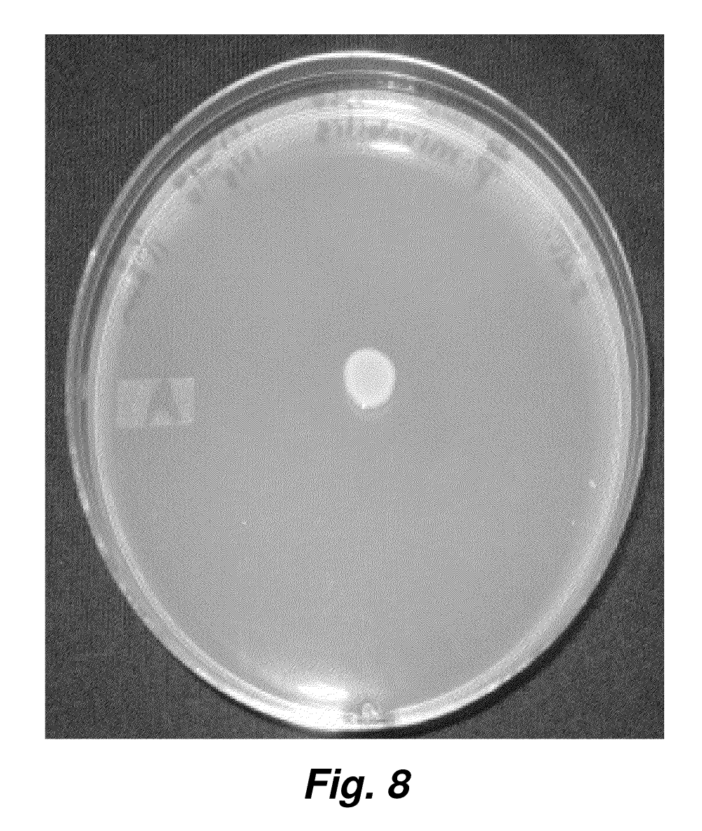

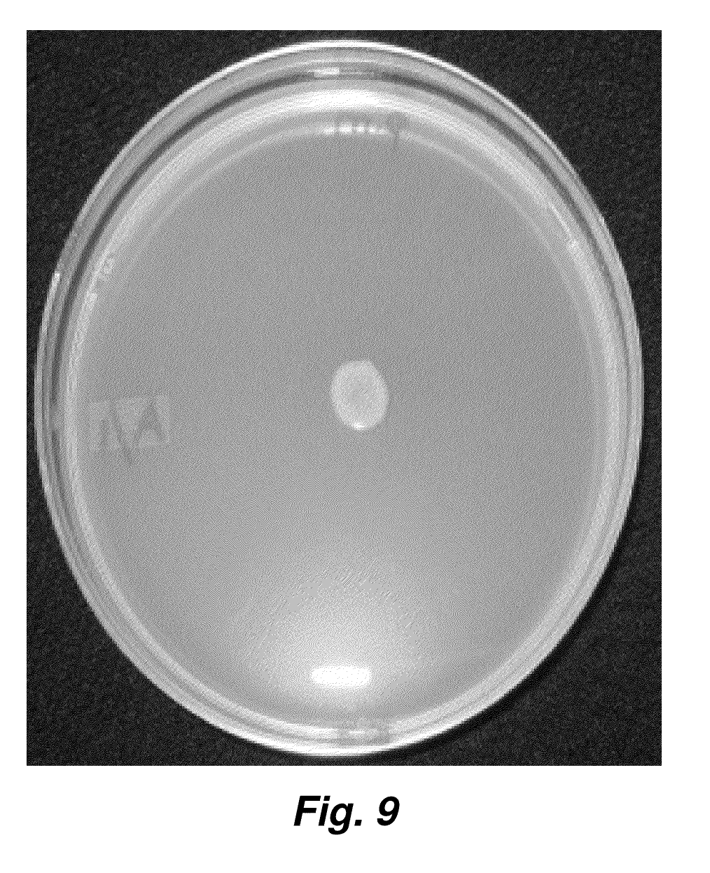

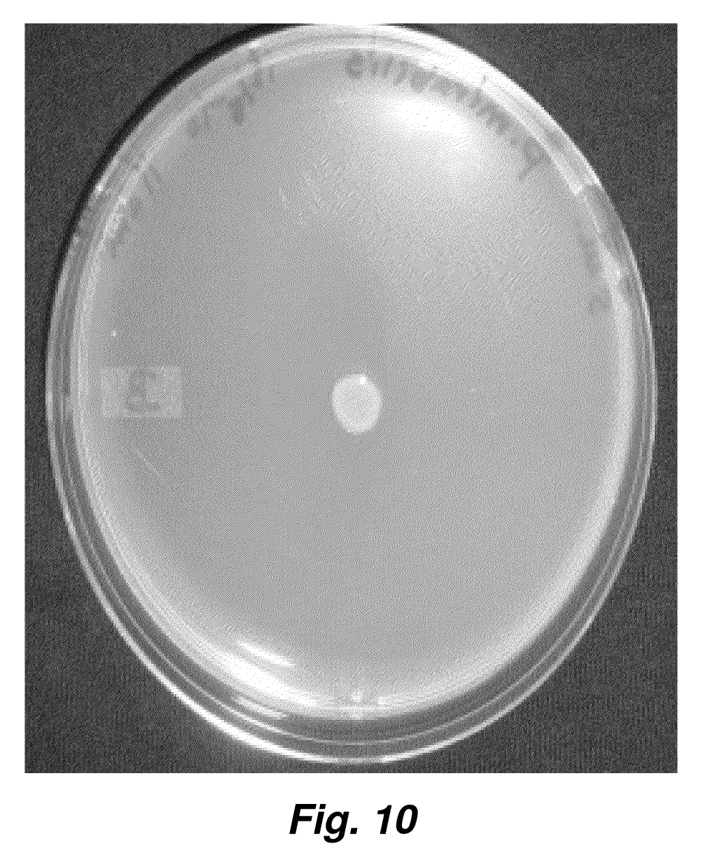

Referring to FIG. 6 through FIG. 10, 5 μl of Proteus mirabilis was inoculated centrally on five separate plates containing a control plate and the four tested culturing media and given 24 hours to grow. FIG. 6 shows an example of swarming tendency on TSA. FIG. 7 shows swarming tendency on cmA-1. FIG. 8 shows swarming tendency on cmA. FIG. 9 shows swarming tendency on cmA/1. FIG. 10 shows swarming tendency on cmB. Swarming is clearly reduced on all tested embodiments.

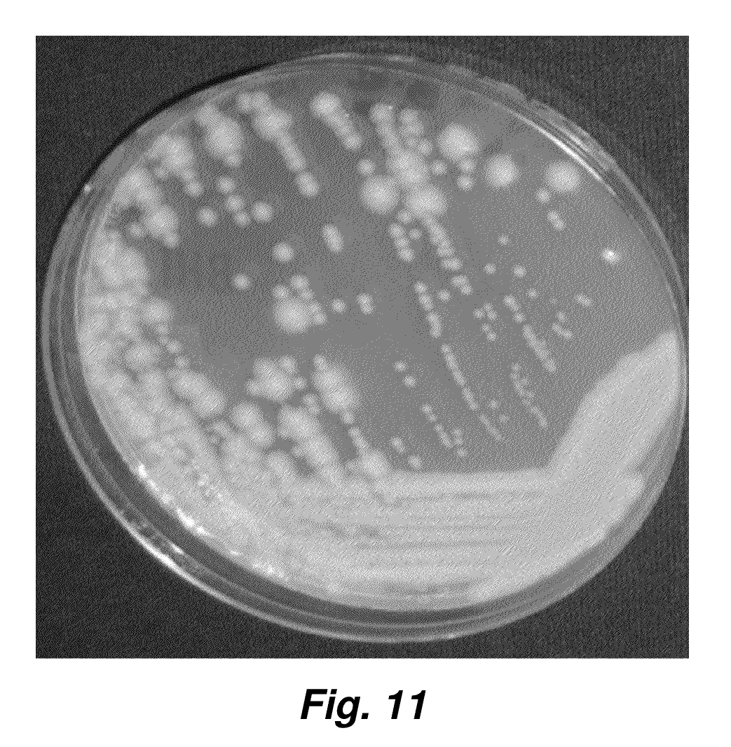

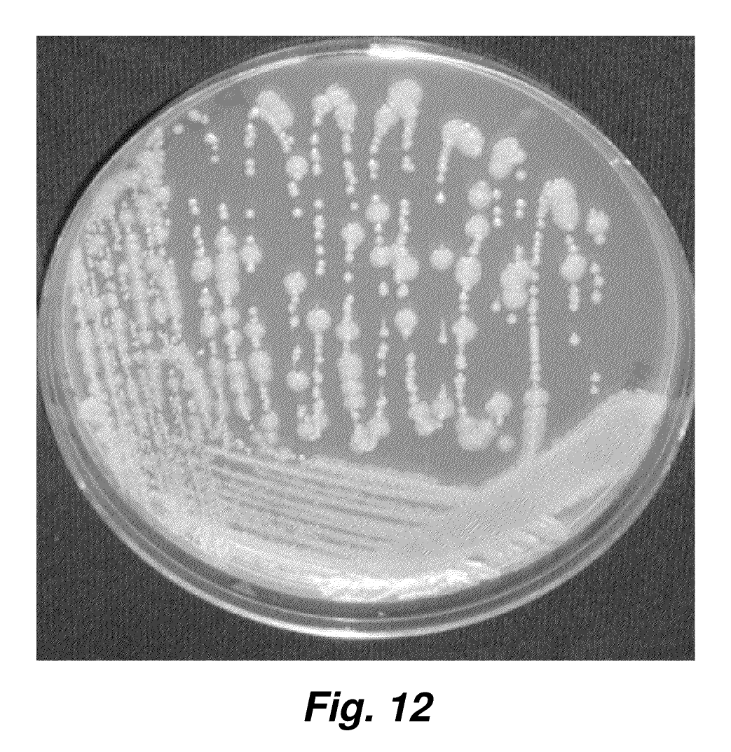

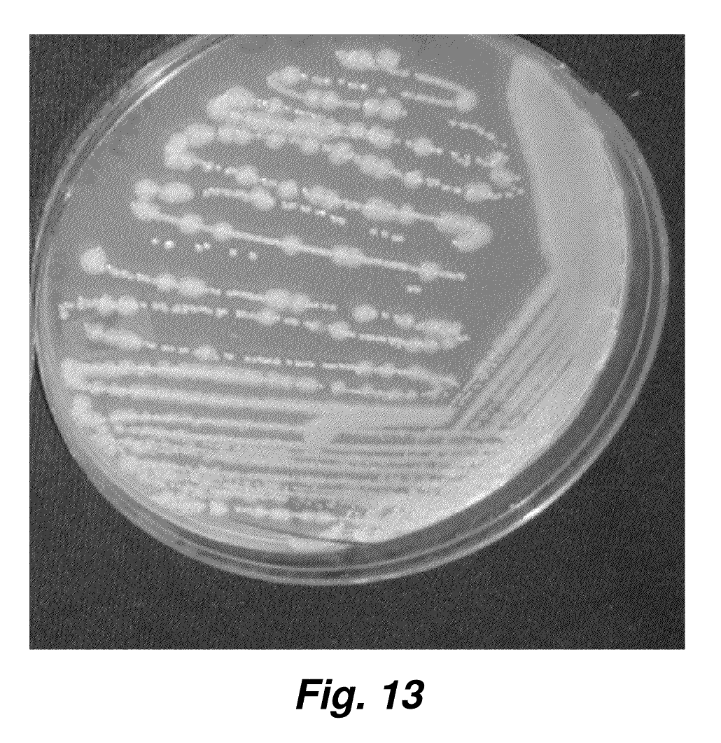

Referring to FIG. 11 through 13, 5 μl of Proteus vulgaris and Bacillus cereus were streak inoculated on three different culturing media plates consisting of a TSA control plate, a cmA/1 plate and an cmA-1 plate. FIG. 11 shows swarming tendency on a TSA control plate. FIG. 12 shows swarming tendency on cmA/1. FIG. 13 shows swarming tendency on cmA-1. Isolation of the different cultures is shown to be greatly increased on the plates containing the tested embodiments.

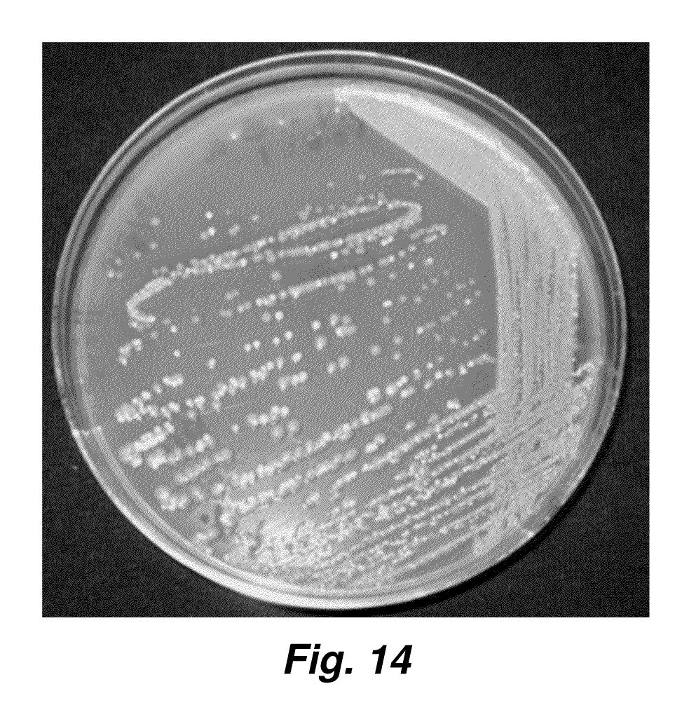

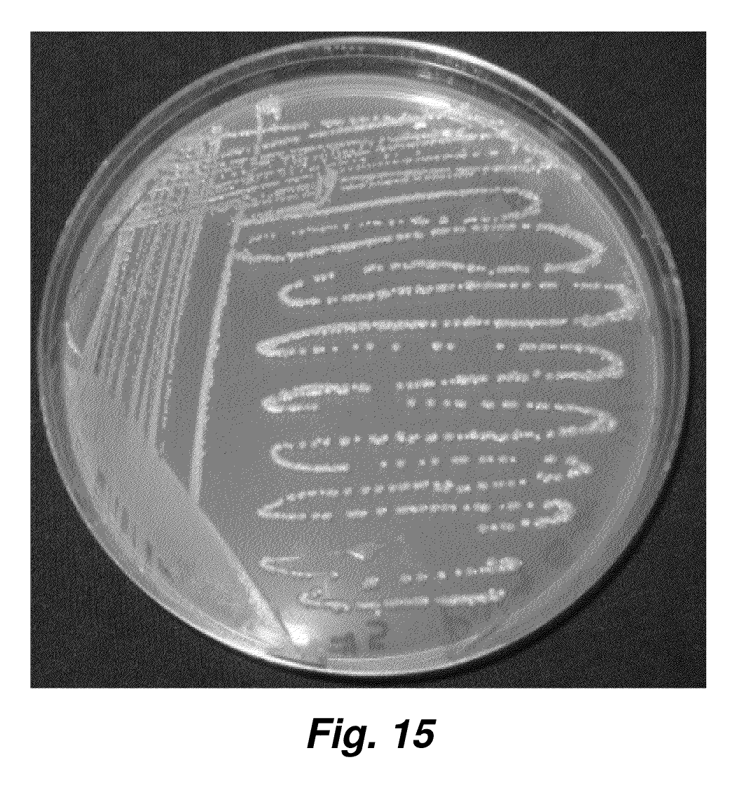

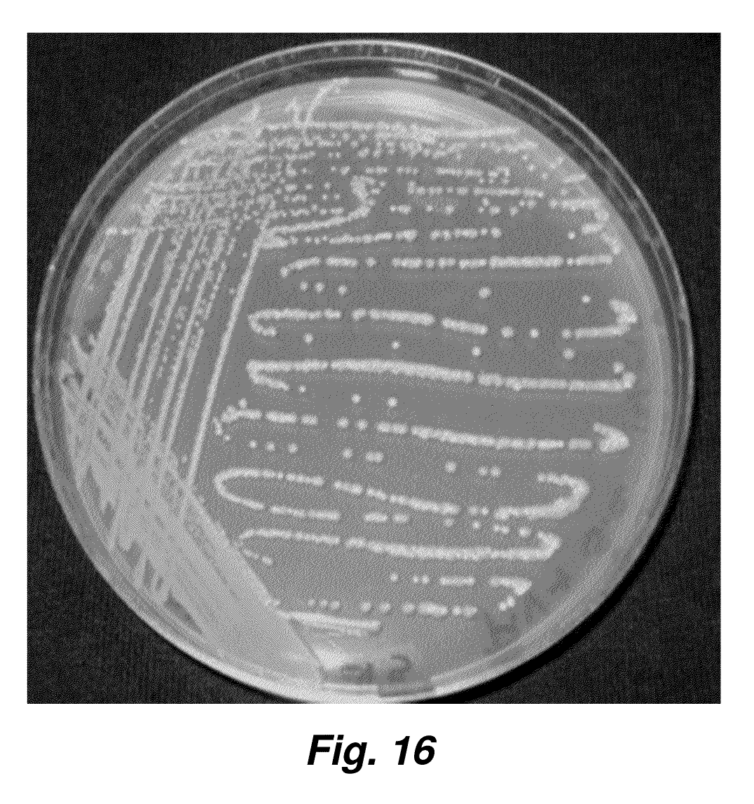

Referring to FIG. 14 through 16, 5 μl of Proteus vulgaris and Staphylococcus aureus were streak inoculated on three different culturing media plates consisting of a TSA control plate, a cmA/1 plate and an cmA-1 plate. FIG. 14 shows swarming tendency on a TSA control plate. FIG. 15 shows swarming tendency on cmA/1. FIG. 16 shows swarming tendency on cmA-1. Isolation of the different cultures is shown to be greatly increased on the plates containing the tested embodiments.

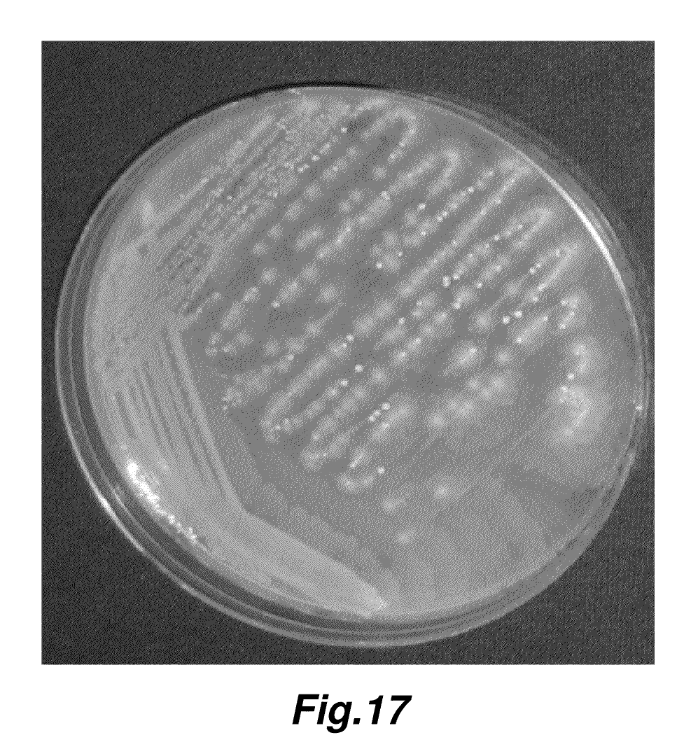

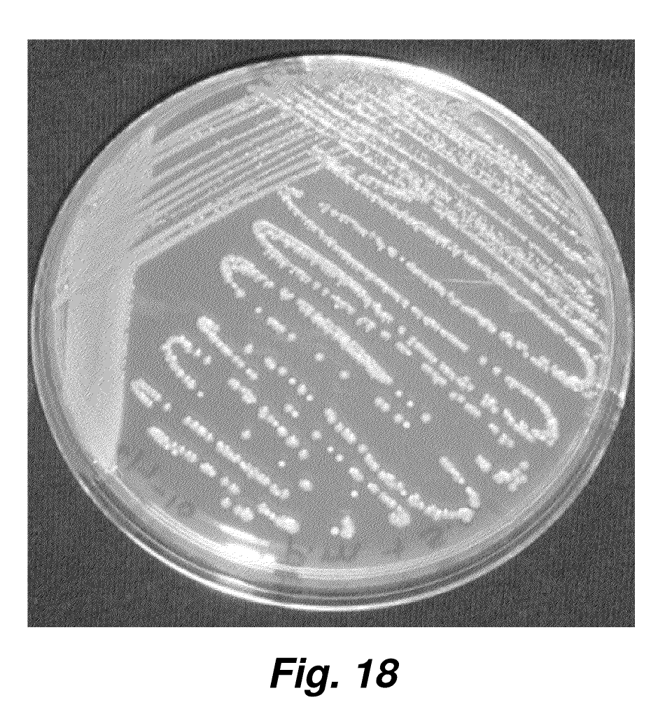

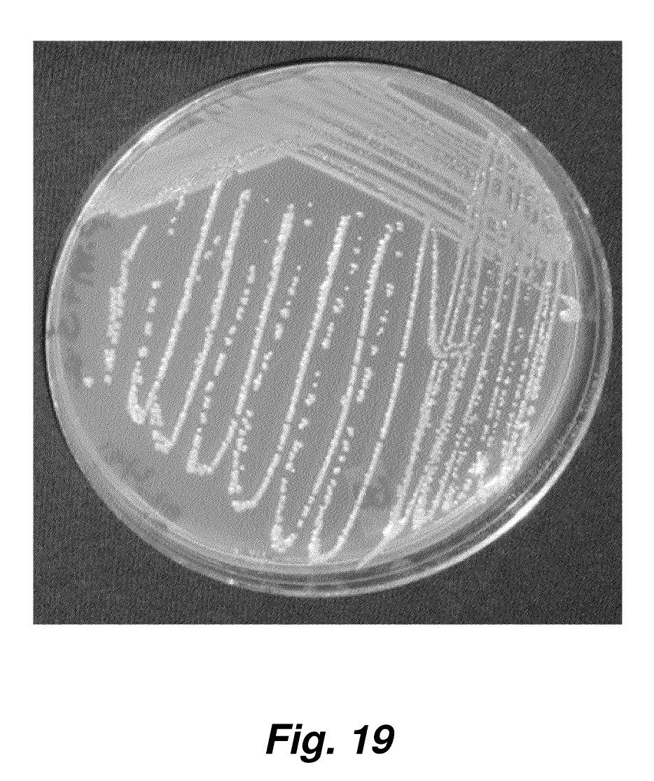

Referring to FIG. 17 through 19, 5 μl of Proteus mirabilis and Staphylococcus aureus were streak inoculated on three different culturing media plates containing a TSA control plate, a cmA/1 plate and an cmA-1 plate. FIG. 17 shows swarming tendency on a TSA control plate. FIG. 18 shows swarming tendency on cmA/1. FIG. 19 shows swarming tendency on cmA-1. Isolation of the different cultures is shown to be greatly increased on the plates containing the tested embodiments.

### Example 2

An Affirmative Approach to Overcome Swarming of Proteus Mirabilis and Proteus Vulgaris.

Introduction: - - - Proteus mirabilis and Proteus vulgaris are gram negative enteric rod shaped bacteria and belong to the genus Proteus and are important pathogens of the Urinary Tract Infection (UTI) in humans.

They are characterized by rapid motility and production of an enzyme called Urease. Individuals suffering from UTI caused by Proteus mirabilis often develop Cystitis, Bacteriurea, Kidney and bladder stones.

Each cell of Proteus has peritrichous flagella which aid in the rapid movement. Because of this function the cells colonies growing on the agar plates often exhibit a characteristic Swarming phenomenon and cover the entire surface making it difficult to form distinct colonies. Cells growing at the edge of the growing colony are more rapidly motile than those in the center; the former move a short distance away from the colony in a mass and then undergo a reduction in motility, settle down as thick or massive zones and divide forming a new crop of motile cells that again swarm and appears as lighter zones. As a result the mature 24 hours colony appears as a series of concentric zones - - - massive alternating with lighter zones.

Because of the swarming of Proteus, bacteriologists and students majoring in microbiology experience hard time, especially when they have to isolate them from an unknown mixture containing more than two different types of bacterial species.

Objective/Purpose: - - - The objective in this example is to formulate a non-selective and non-differential microbiological agar medium that serves two purposes.

1. To control the swarming of Proteus species and Alcaligenes faecalis (found in feces), when they are grown on the new agar plates, thus helping in easy and clear isolation from a mixed culture.

2. To make sure that all types of non-fastidious bacteria used in General Microbiology and Pathogenic Microbiology teaching labs. Grow well on this new agar and exhibit normal results for their biochemical tests.

Methods and Materials:

Bacteria:

Gram Negative Bacteria: - - - Proteus mirabilis ATCC#43071, Proteus vulgaris ATCC#8427, Alcaligenes faecalis, Escherichia coli, Salmonella typhimurium, Salmonella sps. Enterobacter aerogenes, Shigella sonii, Pseudomonas aeroginosa, Serratia marcens, Providencia rettgeri, Citrobacter frundi.

Gram Positive Bacteria: - - - Staphylococcus aureus, Staphylococcus epidermidis, Staphylococcus saprophyticus, Bacillus cereus, Cornybacterium diphtheria(avirulent).

Lab.Wares: - - - 2 Liter flasks, Hot plates, Stir bars, Deionized water, Autoclave, Sterile Petri dishes labeled as “Control, ASAP-I, ASAP-II, ASAP- III, ASAP-IV” (this labeling represents the different compositions used to formulate the new “Anti-Swarming Agar for Proteus”.

Ingredients/Media:

| | |
|---|---|
| BD Bacto Tryptone | Ref. # 211705 |
| BD Bacto Soytone | Ref. # 243620 |
| Bacto Yeast Extract Technical | Ref. # 288620 |
| Difco Agar Technical | Ref. # 281230 |
| Sodium Chloride | Ref. # F-S671-10 |
| Difco Tryptic Soy Broth | Ref. # 211822 |

Procedure : - - - To prepare one liter of “Control, ASAP-I, ASAP-II, ASAP-III, ASAP-IV” agar medium the following ingredients are used.

| | | | | | | | | | Control (Difco. Tryptic Soy Agar) | | |
|---|---|---|---|---|---|---|---|---|---|---|---|
| | ASAP-I | | ASAP-II | | ASAP-III | | ASAP-IV | | (premix) | | |
| Bacto soytone | 5 | grams | 5 | grams | 5 | grams | 5 | grams | Papaic digest of soybean | 5 | g. |
| Bacto Tryptone | 10 | grams | 10 | grams | 12 | grams | 10 | grams | Pancreatic digest of casein | 15 | g. |
| Bacto Yeast Extract | 1 | gram | 5 | grams | --- 0 --- | | 3 | grams | | ---0--- | |
| technical | | | | | | | | | | | |
| Sodium chloride | 15 | grams | 15 | grams | 15 | grams | 15 | grams | Sodium chloride | 5 | g. |
| Difco Agar technical | 25 | grams | 25 | grams | 25 | grams | 25 | grams | Agar | 15 | g. |
| | 56 | g/lit. | 60 | g/lit. | 57 | g/lit. | 58 | g/lit. | | 40 | g/lit. |

Five 2 liter flasks are labeled as ASAP-I, ASAP-II, ASAP-III, ASAP-IV, and Control using an autoclave tape. The above mentioned ingredients are measured very accurately and added to the corresponding flasks each with one liter of deionized water and a stir bar in it. Heated with agitation on a hot plate and boiled for one minute to completely dissolve the ingredients. The flasks plugged with stoppers and covered with aluminum foil. Autoclaved at 1210C for 15 minutes and cooled in a 500C water bath. Then aseptically dispensed in the petri dishes—20 ml/petri dish. The dispensed plates allowed to solidify and left at room temperature at least for 48 hrs before use for better results. Note: - - - Any microbiological agar medium when used within 48 hrs. after dispensing, the moisture in it smears the culture and makes it difficult to isolate the colonies.

Preparation of cultures: - - - To conduct the experiments, Proteus mirabilis, Proteus vulgaris and Alcaligenes faecalis are grown separately in Tryptic Soy Broth #211822 @340C for 20 hrs. 5 ul. of each organism is inoculated centrally as well as using 4-zones streaking technique on all of the above mentioned experimental agar plates and Tryptic Soy Agar plates as a “Control”. All plates incubated for 24 hrs. Results recorded and incubation continued for upto 40 hrs. Pictures taken are showing 40 hrs. incubation results.

Simultaneously a number of mixed cultures are prepared in different combinations (in Tryptic Soy Broth) using i P. mirabilis, P. vulgaris, A. faecalis, and the gram positive and gram negative bacteria from the pre-mentioned list. 5 μl of each mixture is used to inoculate the experimental and control plates, using the 4-zone technique. Pictures are taken only for few combinations.

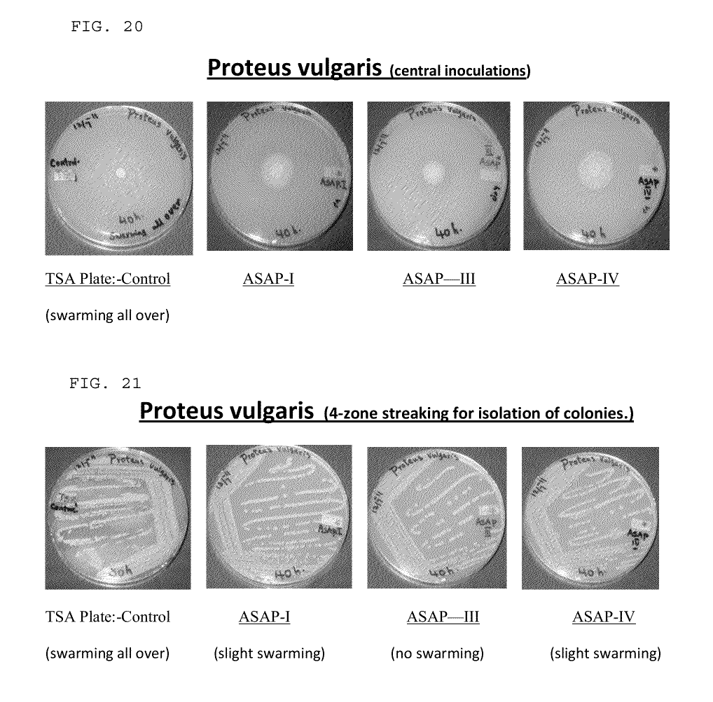

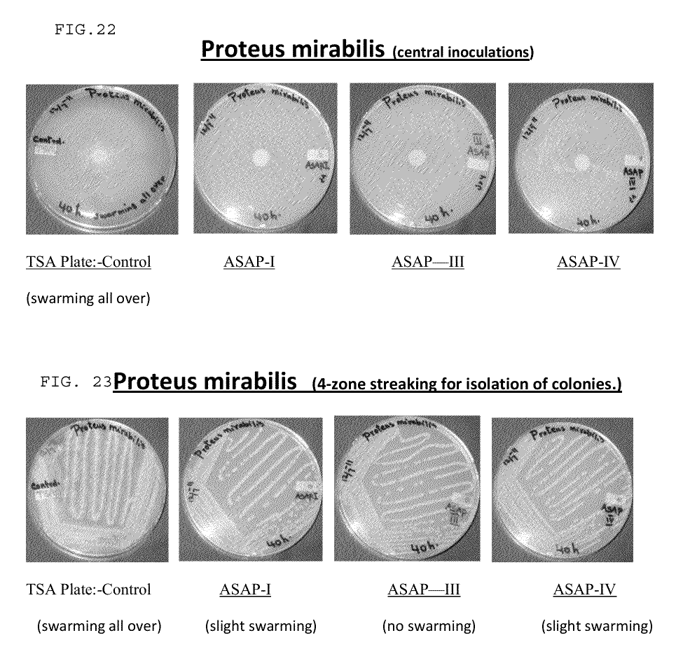

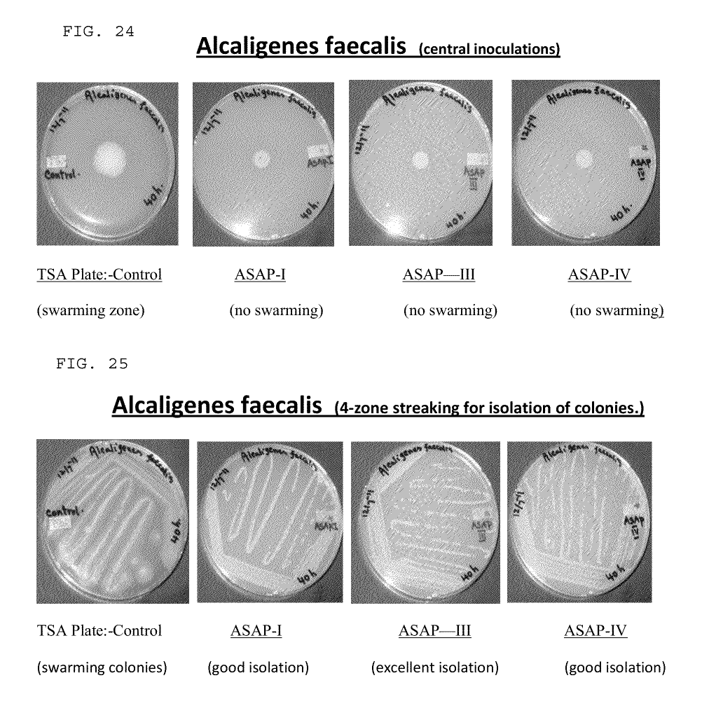

Observations and Results: - - -All plates are checked after 24 hrs. & 40 hrs. For centrally inoculated plates the radius of the swarming zones are measured and recorded in the decreasing order as follows.
P. mirabilis: Tryptic Soy Agar>ASAP II>ASAP IV>ASAP I>*ASAP III (FIGS. 22 and 23)
P. vulgaris: Tryptic Soy Agar>ASAP II>ASAP IV>ASAP I>*ASAP III (FIGS. 20 and 21)
A. faecalis: Tryptic Soy Agar>ASAP II>ASAP IV>ASAP I>*ASAP III (FIGS. 24 and 25).

On the 4-zone streaked plates P. mirabilis, P. vulgaris by themselves and in combination with other gram positive bacteria like Staphylococcus and Bacillus cereus exhibited excellent distinct colonies on plate ASAP III.

Conclusion: - - - Plate “ASAP III” composition worked best to inhibit the Proteus species swarming as well as for the isolation and growth of variety of non-fastidious gram positive and gram negative bacteria.

Name and Formula of the New Agar:

Anti-Swarming Agar for Proteus sps. {ASAP III}

| | |
|---|---|
| BactoSoytone | 5 gr. |
| Bacto Tryptone | 12 gr. |
| Sodium Chloride | 15 gr. |
| Difco Agar Technical | 25 gr. |
| Amount required | 57 gr./Liter of Deionized Water. |

Note:
It was found that Agar Medium works best between 48 hrs. to 3 weeks after dispensing.

While various disclosed embodiments have been described above, it should be understood that they have been presented by way of example only, and not limitation. Numerous changes to the subject matter disclosed herein can be made in accordance with this Disclosure without departing from the spirit or scope of this Disclosure. In addition, while a particular feature may have been disclosed with respect to only one of several implementations, such feature may be combined with one or more other features of the other implementations as may be desired and advantageous for any given or particular application.

Thus, the breadth and scope of the subject matter provided in this Disclosure should not be limited by any of the above explicitly described embodiments. Rather, the scope of this Disclosure should be defined in accordance with the following claims and their equivalents.

The terminology used herein is for the purpose of describing particular embodiments only and is not intended to be limiting. As used herein, the singular forms “a,” “an,” and “the” are intended to include the plural forms as well, unless the context clearly indicates otherwise. Furthermore, to the extent that the terms “including,” “includes,” “having,” “has,” “with,” or variants thereof are used in either the detailed description and/or the claims, such terms are intended to be inclusive in a manner similar to the term “comprising.”

Unless otherwise defined, all terms (including technical and scientific terms) used herein have the same meaning as commonly understood by one of ordinary skill in the art to which embodiments belong. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

What is claimed is,:

1. A culturing media powder, comprising:
at least one agar material;
at least one nutrient; and
a swarming inhibiting quantity of an anti-swarming compound;
wherein the ratio of said at least one agar material relative to the quantity of said anti-swarming compound is such to ameliorate cellular swarming by a first microbe and increasing cellular growth rate of a second microbe relative to a swarm rate of said first microbe when said first and second microbes are grown on a culturing media made of said culturing media powder.

2. The culturing media powder of claim 1, wherein the ratio of said anti-swarming compound to said agar material to said at least one nutrient is about 2.5-3.5: about 4.5-5.5: about 3.5-4.5 respectively.

3. The culturing media powder of claim 1, wherein said anti-swarming compound comprises NaCl and is present in an amount ranging from about 20-40% by weight of said powder and in a ratio of about 1-3.5: about 1-5 of said NaCl to said agar material, respectively.

4. The culturing media powder of claim 1, wherein a weight of powder per volume solution dose (w/v dose) of said culturing media powder is effective to provide said swarming inhibiting quantity of anti-swarming compound ranging from about 1-4% w/v.

5. The culturing media powder of claim 4, wherein said w/v dose is about 60 grams of said culturing media powder per liter of said water solution.

6. The culturing media powder of claim 4, wherein said w/v dose of said culturing media powder is effective to provide said swarming inhibiting quantity of said anti-swarming compound in an amount of about 1.5% w/v of a water solution.

7. The culturing media powder of claim 4, wherein said w/v dose is effective to provide a quantity of said agar material in an amount of about 1.5-3% w/v of said water solution.

8. The culturing media powder of claim 7, wherein said w/v dose is about 60 grams of said culturing media powder per liter of said water solution.

9. The culturing media powder of claim 8, wherein at least one nutrient comprises a papaic digest of soybean and a pancreatic digest of casein, and
wherein a w/v dose is effective to provide said papaic digest of soybean in an amount of about 0.5% w/v of said water solution, said pancreatic digest of casein in an amount of about 1.5% w/v of said water solution, said agar material in an amount of about 2.5% w/v of said water solution, and said anti-swarming compound in an amount of about 1.5% w/v of said water solution.

10. An anti-swarming culturing media slurry, comprising:
at least one agar material;
at least one nutrient;
a quantity of water; and
a swarming inhibiting quantity of an anti-swarming compound wherein the ratio of said at least one agar material relative to the quantity of said anti-swarming compound is such to ameliorate cellular swarming by a first microbe and increasing cellular growth rate of a second microbe relative to a swarm rate of said first microbe when said first and second microbes are grown on a culturing media made of said culturing media slurry.

11. The anti-swarming culturing media slurry of claim 10, wherein said agar material is present in an amount ranging from 15-30 g/L water, and said anti-swarming compound is present in an amount ranging from about 10-40 g/L water.

11. The anti-swarming culturing media slurry of claim 10, wherein said agar material is present in an amount of 25 g/L of water, and said anti-swarming compound is present in an amount of about 15 g/L of water.

12. The anti-swarming culturing media slurry of claim 10, wherein said at least one nutrient comprises papaic digest of soybean in an amount of about 5 g/L of water and pancreatic digest of casein in an amount of about 15g per 1000 ml or water.

13. A culturing apparatus for preventing swarming, comprising:
at least one dish; and
an anti-swarming culturing media disposed on at least a portion of at least one dish made from an anti-swarming culturing media slurry,
said anti-swarming culturing media slurry comprising water, an agar material present in an amount of about 25 g/L of water, papaic digest of soybean in an amount of about 5 g/L of water, pancreatic digest of casein in an amount of about 15 g/L of water, and sodium chloride in an amount of about 15 g/L of water.

14. The culturing media powder of claim 4, wherein said w/v dose of said culturing media powder is effective to provide said swarming inhibiting quantity of said anti-swarming compound in an amount of about 1.5-1.7% w/v of a water solution.

15. A culturing media powder composition, the composition comprising:
at least one agar material;
at least one nutrient; and
a swarming inhibiting quantity of an anti-swarming compound; wherein said anti-swarming compound is about 26%, by weight, of said composition.16. The culturing media powder composition of claim 15, wherein said at least one agar material is about 44%, by weight, of said composition.

17. The culturing media powder composition of claim 15, wherein said at least one nutrient is about 30%, by weight, of said composition.

18. A culturing apparatus for preventing swarming, comprising:
at least one dish; and
an anti-swarming culturing media disposed on at least a portion of at least one dish made from an anti-swarming culturing media slurry,
said anti-swarming culturing media slurry comprising water and an amount of the composition of claim 15, wherein said amount is about 6% w/v.

19. The culturing apparatus of claim 18, wherein said agar material is about 2.6% w/v of said slurry and said at least one nutrient is about 1.8% of said slurry.

20. The culturing apparatus of claim 18, wherein said anti-swarming compound is about 1.56% w/v.
